(19) United States
(12) Patent Application Publication
(10) Pub. No.: US 2005/0159334 A1
(43) Pub. Date: Jul. 21, 2005

(54) Method for treating rheumatoid arthritis by inhibiting peptidylarginine deiminase

(76) Inventors: Oscar S. Gluck, Phoenix, AZ (US); Michael J. Maricic, Tucson, AZ (US)

Correspondence Address: WEISS & MOY PC, 4204 NORTH BROWN AVENUE, SCOTTSDALE, AZ, 85251, US

(21) Appl. No.: 10/759,881

(22) Filed: Jan. 16, 2004

(51) Int. Cl. A61K 38/54

(52) U.S. Cl. 514/2

(57) ABSTRACT

Rheumatoid arthritis is treated with the administration of a therapeutic dose of a therapeutically acceptable PAD inhibitor. Administration can occur after the onset of RA symptoms, or prophylactically before such symptoms present. In one embodiment, the inhibitor has a side chain including a benzamide group to the left and an ester group to the right of a peptide bond.

### FIELD OF THE INVENTION

This invention relates generally to treatments and methods for treating and/or preventing rheumatoid arthritis and, more particularly, to a method for treating (including in one embodiment preventing the onset of) rheumatoid arthritis through the inhibition of peptidylarginine deiminase.

### BACKGROUND OF THE INVENTION

Rheumatoid arthritis (RA) is a debilitating, chronic inflammatory disease affecting 1 to 2% of the world's population. It afflicts approximately 2.1 million people in the United States.

RA causes pain, swelling and destruction of multiple joints. Fifty percent of patients become disabled within 5 years and the standardized mortality rate for rheumatoid arthritis patients is twice that of the standard population. Complications such as Sjogren's syndrome, vasculitis, pericarditis an increased incidence of lymphoma and infections are common in rheumatoid arthritis patients.

RA is an autoimmune disease. Its initiating factor is presently unknown, and diagnosis can be difficult in the disease's early stages. In RA, white blood cells attack the synovium, causing inflammation known as synovitis. It results in warmth, redness, swelling and pain—symptoms that are typical of RA. During the inflammation process, the cells of the synovium grow and divide abnormally, making the normally thin synovium thick and resulting in a joint that is swollen and puffy.

Peptidylarginine deiminase (PAD) is an 85 kilo-Dalton, calcium-dependent enzyme that catalyzes the deimination of Peptidylarginine to form Peptidylcitrulline. Vossenar E R, Zendman A S W, vanVenrooij W J, Pruijn G J M. PAD, a growing family of citrullinated enzymes: genes, features and involvement in disease. BioEssays 2003; 25:116-1118. PAD has been found in many tissues, including epidermis, skeletal muscle, brain and synovium. Deimination of arginyl residues can affect protein structure, protein-protein interactions and the activities of various enzymes.

There are several isotypes of PAD. In the inflammatory RA, PAD 2 and PAD 4 are present in large quantities, where they catalyze the production of significant amounts of citrullinated fibrin. These citrullinated proteins in the synovium are the major autoantigens driving the local immune response, suggested by the discovery of local production of anti-citrulline antibodies in the synovial tissue of patients with RA. A recent study has shown highly statistically significant linkage disequilibrium with the isoenzyme PAD type 4 with the development of rheumatoid arthritis in humans. Suzuki A, Yamada R, Chang X. Functional haplotypes of PADI4 encoding peptidylarginine deiminase enzyme 4 are associated with rheumatoid arthritis. Nature Genetics 2003; 34:4.395-403. In addition, it has been recently shown that citrullination of synovial proteins takes place in both the strep cell wall and collagen-induced arthritis mouse models. Vossenaar E R, Nijenhuis S, van Helsen M, et al. Citrullination of synovial proteins in murine models of rheumatoid arthritis. Arthritis Rheum 2003; 48:9.S348. In both models, PAD isoenzyme type 4, although absent from healthy synovia, is transcribed and translated by polymorphonuclear leukocytes infiltrating inflamed synovial tissue.

Proteins may undergo posttranslational modification either to carry out a particular functional role or to allow recycling of amino acids. Zhou Z, MD, Ménard H A. Autoantigenic posttranslational modifications of proteins: does it apply to rheumatoid arthritis? Curr Opin Rheum 2002;14 :250-253. An important feature of the modified proteins is the acquisition of autoantigenicity. One example of posttranslational modification of a protein that plays an autoantigenic role in rheumatoid arthritis is modification of immunoglobulin G that becomes a target for rheumatoid factors. Citrullination or the deimination of arginine residues in proteins creates epitopes that are targeted by rheumatoid autoantibodies.

Rheumatoid arthritis is genetically associated with Major Histocompatability Complex (MHC) class II molecules that contain a shared epitope. The conversion of arginine to citrulline at the peptide side-chain position interacts with the shared epitope and significantly increases peptide-MHC affinity leading to activation of CD4(+) T cells in DR4+ mice. Hill J A, Southwood S, Sette A, Jevnikar A M, Bell D A, Cairns E. Cutting edge: the conversion of arginine to citrulline allows for a high-affinity peptide interaction with the rheumatoid arthritis-associated HLA-DRB1*0401 MHC class II molecule. J. Immunol. Jul. 15, 2003; 171(2):538-41.

Citrullinated proteins fit stereochemically into the HLA-DR4 antigen binding grooves. HLA-DR4 is the most common genetic haplotype found in patients with RA, and approximately 80% of RA patients have the HLA-DR4 gene. This suggests that citrullinated proteins are a pivotal initial stimulus for the immune dysregulation in RA.

Anti-cyclic citrullinated peptide (anti-CCP) antibodies are present in the majority of patients with RA within the first year of disease onset, further confirming the role of citrullinated proteins in the initiation of the immune dysregulation of RA. In fact, in one study, anti-CCP could be detected up to 2.6 years before the clinical onset of rheumatoid arthritis. Berglin E, Padyukov L Hallmans G, et al et al. Arthritis Rheum 2003; 48:9. S678. The specificity of anti-CCP antibodies in RA is 98%. A study using the CCP2 assay (a second generation assay) found progression from undifferentiated polyarthritis to RA in 93% of anti-CCP positive patients but only in 25% of anti-CCP negative patients after 3 years of follow-up. Rheumatoid factor and antibodies to cyclic citrullinated Jansen A L et al. Peptide differentiate rheumatoid arthritis from undifferentiated polyarthritis in patients with early arthritis. J. Rheumatol 2002; 29:2074-6.

This evidence suggests a key pathological role of citrullinated proteins in the genesis of RA. The present invention concerns the inhibition of the isoenzymes of PAD, in order to block the citrullination of proteins pivotal to the initiation and progression of RA.

### SUMMARY OF THE INVENTION

In accordance with one embodiment of the present invention, a method for treating a host for rheumatoid arthritis is disclosed. The method comprises administering to a host a composition including a therapeutic dose of a therapeutically acceptable PAD inhibitor.

### BRIEF DESCRIPTION OF THE DRAWINGS

FIG. 1 is an illustration of a generic PAD inhibitor consistent with an embodiment of the present invention.

### DETAILED DESCRIPTION OF THE PREFERRED EMBODIMENTS

Generally, the nomenclature used hereafter, and the laboratory procedures in cell culture and protein biochemistry are those well known and commonly employed in the art. The terms “pharmaceutically acceptable” or “therapeutically acceptable” refer to a substance which does not interfere with the effectiveness or the biological activity of the active ingredients and which is not toxic to the host or the patient.

As used herein, “therapeutic dose” is a dose which prevents, alleviates, abates, or otherwise reduces the severity of RA symptoms in a patient. The compositions of the invention may be used prophylactically to prevent the onset of RA or may be therapeutically used after the onset of symptoms.

The quantities of active ingredient necessary for effective therapy will depend on many different factors, including means of administration, target site, physiological state of the patient, and other medicaments administered. Thus, treatment dosages should be titrated to optimize safety and efficacy. Typically, dosages used in vitro may provide useful guidance in the amounts useful for in situ administration of the active ingredients. Animal testing of effective doses for treatment of particular disorders will provide further predictive indication of human dosage. Various considerations are described, for example, in Goodman and Gilman's The Pharmacological Basis of Therapeutics, 7th Edition (1985), MacMillan Publishing Company, New York, and Remington's Pharmaceutical Sciences 18th Edition, (1990) Mack Publishing Co, Easton, Pa. Methods for administration are discussed therein, including oral, intravenous, intraperitoneal, intramuscular, transdermal, nasal, iontophoretic administration, and the like.

The compositions of the invention may be administered in a variety of unit dosage forms depending on the method of administration. For example, unit dosage forms suitable for oral administration include solid dosage forms such as powder, tablets, pills, capsules, and dragees, and liquid dosage forms, such as elixirs, syrups, and suspensions. The active ingredients may also be administered parenterally in sterile liquid dosage forms. Gelatin capsules contain the active ingredient and as inactive ingredients powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The concentration of the compositions of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The compositions of the invention may also be administered via liposomes. Liposomes include emulsions, foams, micelles, insoluble monolayers, liquid crystals, phospholipid dispersions, lamellar layers and the like. In these preparations the composition of the invention to be delivered is incorporated as part of a liposome, alone or in conjunction with a molecule which binds to a desired target, such as antibody, or with other therapeutic or immunogenic compositions. Thus, liposomes either filled or decorated with a desired composition of the invention of the invention can delivered systemically, or can be directed to a tissue of interest, where the liposomes then deliver the selected therapeutic/immunogenic polypeptide compositions.

Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. Ann. Rev. Biophys. Bioeng. 9:467 (1980), U.S. Pat. Nos. 4,235,871, 4,501,728, 4,837,028, and 5,019,369, incorporated herein by reference.

A liposome suspension containing a composition of the invention may be administered intravenously, locally, topically, etc. in a dose which varies according to, inter alia, the manner of administration, the composition of the invention being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more compositions of the invention of the invention, and more preferably at a concentration of 25%-75%.

For aerosol administration, the compositions of the invention are preferably supplied in finely divided form along with a surfactant and propellant. Typical percentages of compositions of the invention are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

The constructs of the invention can additionally be delivered in a depot-type system, an encapsulated form, or an implant by techniques well-known in the art. Similarly, the constructs can be delivered via a pump to a tissue of interest.

Any of the foregoing formulations may be appropriate in treatments and therapies in accordance with the present invention, provided that the active agent in the formulation is not inactivated by the formulation and the formulation is physiologically compatible.

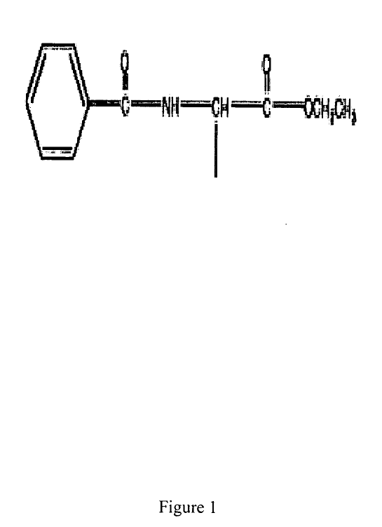

The present invention concerns treatments and therapies for preventing the onset of RA and/or amerliorating its symptoms. In one embodiment, a therapeutic dose of a PAD inhibitor is administered. Referring now to FIG. 1, an illustration of a generic PAD inhibitor is provided. As shown in FIG. 1, in this embodiment, a PAD inhibitor will have a side chain including a benzamide group to the left and an ester group to the right of a peptide bond. Known PAD inhibitors contain this chemical structure, which has been shown to bind to PAD, inhibiting the production of citrullinated proteins. See, e.g., Prizker L B, Moscarello M A. A novel microtubule independent effect of paclitaxel: the inhibition of peptidylarginine deiminase from bovine brain. Biochimica et Biophysica Acta 1388 (1998) 154-160. Modification of known PAD inhibitors, to reduce or eliminate toxicity, utilizing techniques known in the art, is appropriate. In addition, other PAD inhibitors, having different structures, may be identified and utilized for purposes of the methods claimed herein.

It should be emphasized that PAD inhibitors can be administered after the onset of RA, to alleviate its symptoms. Alternatively, PAD can be administered phrophylactically. For example, persons having the HLA-DR4 gene, including those who are particularly within a risk category of RA (e.g., female sex, between the ages of 25 and 50), may be appropriate candidates for such preventive treatment.

All publications and patent applications cited in this specification are herein incorporated by reference in their entirety as if each individual publication or patent application were specifically and individually indicated to be incorporated by reference.

As will be apparent to those skilled in the art to which the invention pertains, the present invention may be embodied in forms other than those specifically disclosed above, without departing from the spirit or essential characteristics of the invention. The particular embodiments of the invention described above, are, therefore to be considered as illustrative and not restrictive. The scope of the present invention is as set forth in the appended claims rather than being limited to the examples contained in the foregoing description.

1. A method for treating a host for rheumatoid arthritis, the method comprising:
administering to a host a composition including a therapeutic dose of a therapeutically acceptable PAD inhibitor.

2. The method of claim 1 wherein said administering step occurs after the onset of symptoms of rheumatoid arthritis.

3. The method of claim 1 wherein said administering step occurs before the onset of symptoms of rheumatoid arthritis.

4. The method of claim 1 wherein said PAD inhibitor has a side chain including a benzamide group to the left and an ester group to the right of a peptide bond.

5. The method of claim 4 wherein said administering step occurs after the onset of symptoms of rheumatoid arthritis.

6. The method of claim 4 wherein said administering step occurs before the onset of symptoms of rheumatoid arthritis.
